(19) United States
(12) Patent Application Publication
(10) Pub. No.: US 2023/0380697 A1
(43) Pub. Date: Nov. 30, 2023

(54) HEMODYNAMIC SENSOR-BASED SYSTEM FOR AUTOMATED PREDICTION OF A POST-INDUCTION HYPOTENSIVE EVENT

(71) Applicant: EDWARDS LIFESCIENCES CORPORATION, Irvine, CA (US)

(72) Inventors: Sai Prasad Buddi, Costa Mesa, CA (US); Emmanuel Akpan, Huntington Beach, CA (US); Zhongping Jian, Irvine, CA (US); Feras Al Hatib, Irvine, CA (US); Jacobus Jozef Gerardus Maria Settels, De Hoef (NL); Christine Lee, Long Beach, CA (US)

(21) Appl. No.: 18/361,346

(22) Filed: Jul. 28, 2023

Related U.S. Application Data
(63) Continuation of application No. PCT/US2022/013912, filed on Jan. 26, 2022.
Application No. PCT/US2022/013912 is a continuation of application No. PCT/US2022/013909, filed on Jan. 26, 2022.
Application No. PCT/US2022/013909 is a continuation of application No. PCT/US2022/013906, filed on Jan. 26, 2022.
(60) Provisional application No. 63/144,424, filed on Feb. 1, 2021.
(60) Provisional application No. 63/144,424, filed on Feb. 1, 2021.
(60) Provisional application No. 63/144,424, filed on Feb. 1, 2021.

(51) Int. Cl. A61B 5/02; A61B 5/00; G16H 50/30

(52) U.S. Cl. CPC A61B 5/02028; A61B 5/7275; A61B 5/6826; G16H 50/30; A61B 5/742; A61B 5/0022

(57) ABSTRACT

A method for determining a post-induction score that represents a prediction that a patient will experience a hypotensive event after beginning an administration of anesthesia is disclosed herein that includes receiving, by a hemodynamic monitor prior to the administration of anesthesia on the patient, sensed hemodynamic data representative of an arterial pressure waveform of the patient. The method further includes extracting, by the hemodynamic monitor, at least one waveform feature from the sensed hemodynamic data. Additionally, the method includes determining, by the hemodynamic monitor based on the at least one waveform feature, the post-induction score that represents the likelihood that the patient will experience a hypotensive event after beginning the administration of anesthesia. Finally, the post-induction score is displayed.

### RELATED APPLICATIONS

This application claims priority to the PCT Application having International Application No. PCT/US2022/013912, filed Jan. 26, 2022, and entitled “HEMODYNAMIC SENSOR-BASED SYSTEM FOR AUTOMATED PREDICTION OF A POST-INDUCTION HYPOTENSIVE EVENT”; to the PCT Application having International Application No. PCT/US2022/013909, filed Jan. 26, 2022, and entitled “TRAINING A PREDICTIVE RISK MODEL FOR AUTOMATED PREDICTION OF A POST-INDUCTION HYPOTENSIVE EVENT”; and to the PCT Application having International Application No. PCT/US2022/013906, filed Jan. 26, 2022, and entitled “AUTOMATED PREDICTION OF A POST-INDUCTION HYPOTENSIVE EVENT.” All three of the above-identified PCT applications in turn claim priority to U.S. Provisional Patent Application Ser. No. 63/144,424, filed Feb. 1, 2021 and entitled “PREDICTION OF A POST-INDUCTION HYPOTENSIVE EVENT.” The disclosures of the above patent applications are hereby incorporated by reference in their entirety.

### FIELD OF THE INVENTION

The present disclosure relates generally to arterial blood pressure monitoring and, more specifically, to the prediction of a hypotensive event that may occur after the beginning of administration of anesthesia.

### BACKGROUND

Hypotension, or low blood pressure, can be a harbinger of serious medical complications, and even mortality, for patients under the influence of anesthesia, undergoing surgery, and/or receiving treatment in an intensive care unit (ICU). The dangers associated with the occurrence of hypotension in a patient are due both to the potential injury caused by the hypotension itself and to the many serious underlying medical disorders that the occurrence of hypotension may signify and/or intensify.

In and of itself, hypotension in patients under the influence of anesthesia, surgical patients, or critically ill patients is a serious medical condition. For example, at the beginning of administration/induction of general anesthesia before surgery, a hypotensive event is prevalent. This transient hypotension has been shown to have adverse effects on patient outcome. Further, this period is also prone to decreased vigilance among anesthesia practitioners with regard to hemodynamic changes in the patient. In another example, in the operating room (OR) setting, hypotension during surgery is associated with increased mortality and organ injury. Even short durations of extreme hypotension during surgery are associated with acute kidney injury and myocardial injury. Among critically ill patients, in-hospital mortality may be nearly doubled for patients experiencing hypotension after emergency intubation. For patients under the influence of anesthesia, surgical patients, and seriously ill patients alike, hypotension, if not corrected, can impair organ perfusion, resulting in irreversible ischemic damage, neurological deficit, cardiomyopathy, and renal impairment.

In addition to posing serious risks to patients under the influence of anesthesia, surgical patients, and critically ill patients in its own right, hypotension can be a symptom of one or more other serious underlying medical conditions. Examples of underlying conditions for which hypotension may serve as an acute symptom include sepsis, myocardial infarction, cardiac arrhythmia, pulmonary embolism, hemorrhage, dehydration, anaphylaxis, acute reaction to medication, hypovolemia, insufficient cardiac output, and vasodilatory shock. Due to its association with such a variety of serious medical conditions, hypotension is relatively common, and is often seen as one of the first signs of patient deterioration prior to surgery, in the OR, and in the ICU.

Conventional patient monitoring for hypotension after the beginning of administration/induction of anesthesia but before surgery and in the OR and ICU settings can include continuous or periodic blood pressure measurement. However, such monitoring, whether continuous or periodic, typically provides no more than a real-time assessment. As a result, hypotension in a patient under the influence of anesthesia, in a surgical patient, or critically ill patient is usually detected only after it begins to occur, so that remedial measures and interventions are not initiated until the patient has entered a hypotensive state. Although, as noted above, extreme hypotension can have potentially devastating medical consequences quite quickly, even relatively mild levels of hypotension can herald or precipitate cardiac arrest in patients with limited cardiac reserve.

In view of the frequency with which hypotension is observed to occur after the beginning of administration/induction of anesthesia but before surgery (i.e., in patients under the influence of anesthesia), and due to the serious and sometimes immediate medical consequences that can result when it does occur, a solution enabling prediction of a post-induction hypotensive event, before its occurrence, is highly desirable. However, due to the unpredictable nature of a patient's response to the administration of anesthesia, the accurate prediction of a post-induction hypotensive event provides many challenges.

### SUMMARY

An example method for determining a post-induction score that represents a prediction that a patient will experience a hypotensive event after beginning an administration of anesthesia is disclosed herein that includes receiving, by a hemodynamic monitor prior to the administration of anesthesia on the patient, sensed hemodynamic data representative of an arterial pressure waveform of the patient. The method further includes extracting, by the hemodynamic monitor, at least one waveform feature from the sensed hemodynamic data. Additionally, the method includes determining, by the hemodynamic monitor based on the at least one waveform feature, the post-induction score that represents the likelihood that the patient will experience a hypotensive event after beginning the administration of anesthesia. Finally, the post-induction score is displayed.

An example method for use by a system for training a predictive risk model to determine a post-induction score that represents a prediction that a patient will experience a hypotensive event after beginning an administration of anesthesia is disclosed herein with the system including a hardware processor and predictive risk model training software code stored in a system memory with the hardware processor configured to execute the predictive risk model training software code. The method includes receiving hemodynamic data representing an arterial pressure waveform of a positive subject of a population of subjects including positive subjects that experienced a hypotensive event after beginning the administration of anesthesia and defining hemodynamic data sets for use in training the predictive risk model with the hemodynamic data sets including the arterial pressure waveform collected before the beginning of the administration of the anesthesia from the positive subject. Further, the method includes extracting, by the predictive risk model training software code executed by the hardware processor, waveform features from the arterial pressure waveform of the positive subject and transforming, by the predictive risk model training software code executed by the hardware processor, the waveform features from the positive subject to a first plurality of parameters characterizing the waveform features. Finally, the method includes identifying, from the first plurality of parameters, a predictive set of parameters enabling prediction that the patient will experience the hypotensive event after beginning the administration of anesthesia and computing predictive risk model coefficients to minimize an error of the post-induction score output by the predictive risk model, thereby training the predictive risk model.

An example system for determining a post-induction score that represents a prediction that a patient will experience a hypotensive event after beginning an administration of anesthesia is disclosed herein that includes a hemodynamic sensor that collects sensed hemodynamic data representative of an arterial pressure waveform of the patient, the sensed hemodynamic data being collected from the patient prior to the administration of anesthesia, a system memory that stores post-induction hypotension prediction software code including a hypotension predictive risk model, and a hemodynamic monitor that includes a hardware processor that is configured to execute the post-induction hypotension prediction software code. The post-induction hypotension prediction software is configured to receive the sensed hemodynamic data from the hemodynamic sensor, extract at least one waveform feature from the sensed hemodynamic data, and determine, based on the at least one waveform feature, the post-induction score that represents the likelihood that the patient will experience a hypotensive event after beginning the administration of anesthesia by utilizing the hypotension prediction risk model.

### BRIEF DESCRIPTION OF THE DRAWINGS

FIG. 1 is a perspective view of an example hemodynamic monitor that includes a hardware processor that predicts a likelihood that a patient will experience a post-induction hypotensive event.

FIG. 2 is a perspective view of an example minimally invasive pressure sensor for sensing hemodynamic data representative of arterial pressure of a patient.

FIG. 3 is a perspective view of an example non-invasive sensor for sensing hemodynamic data representative of arterial pressure of a patient.

FIG. 4 is a block diagram illustrating an example hemodynamic monitoring system that determines a likelihood that a patent will experience a post-induction hypotensive event based on hemodynamic data that is collected prior to the beginning of administration of anesthesia.

FIG. 5 is a graph illustrating an example trace of an arterial pressure waveform including example indicia corresponding to the likelihood that a patient will experience a post-induction hypotensive event.

FIG. 6 is a diagram illustrating an example system for training a post-induction hypotensive prediction model.

FIG. 7 is a diagram illustrating another example system for training a post-induction hypotensive prediction model.

FIG. 8 is a diagram illustrating an example system and computer-readable medium including instructions for performing post-induction predictive risk model training.

### DETAILED DESCRIPTION

As described herein, a system for determining a post-induction score, such as a hemodynamic monitoring system, and related methods of determining the post-induction score and training a post-induction predictive risk model to determine the post-induction score produce a post-induction score that represents the likelihood that a patient will experience a hypotensive event after the beginning of administration of anesthesia. The post-induction score is determined based on hemodynamic data, such as arterial pressure waveform data collected by an arterial pressure sensor, during a period of time before the patient is administered/induced anesthesia. The post-induction score is determined and conveyed to a medical professional, such as an anesthesiologist, before the anesthesia is administered so that the medical professional has a warning that the patient is likely (or not likely) to experience a hypotensive event after the beginning of administration of anesthesia. The distinction that the system and methods predict the likelihood that the patient will experience a hypotensive event after the beginning of administration of anesthesia is important because other predictive systems and methods cannot accurately predict the likelihood of a hypotensive event when the patient is under the influence of anesthesia due to the generally unpredictable nature of the patient's response to the anesthesia.

The post-induction score is determined based on a weighted combination of a plurality of hypotension parameters, for example waveform features extracted from the hemodynamic data and/or patient demographic information, that are predictive of the future hypotensive event. Risk coefficients that implement the weighting are selected based on a mean arterial pressure (MAP) threshold for hypotension, such as a pressure of 65 millimeters of Mercury (mmHg) or other defined pressure threshold. The selection of risk coefficients and/or hypotension parameters can be accomplished via training (e.g., offline training) of the post-induction predictive risk model using machine learning or other techniques to minimize the error of the predictive risk model output to the true value of training subsets that define hypotension according to the MAP threshold for hypotension. The post-induction predictive risk model can, for example, define a hypotensive event as the mean arterial pressure of the patient dropping below 65 millimeters Mercury (mmHg) for at least one minutes within a period of fifteen minutes from the time anesthesia is starting to be administered. Other definitions of what constituents a hypotensive event can be utilized by the post-induction predictive risk model.

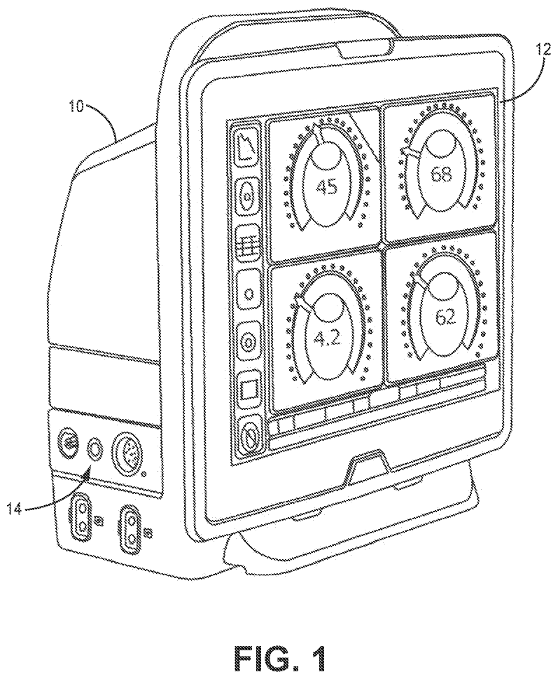

FIG. 1 is a perspective view of hemodynamic monitor 10 that determines a post-induction score representing a likelihood that a patient will experience a future hypotensive event after the beginning of administration/induction of anesthesia. As illustrated in FIG. 1, hemodynamic monitor 10 includes display 12 that, in the example of FIG. 1, presents a graphical user interface including control elements (e.g., graphical control elements) that enable user interaction with hemodynamic monitor 10. Hemodynamic monitor 10 can also include a plurality of input and/or output (I/O) connectors configured for wired connection (e.g., electrical and/or communicative connection) with one or more peripheral components, such as one or more hemodynamic sensors, as is further described below. For instance, as illustrated in FIG. 1, hemodynamic monitor 10 can include I/O connectors 14. While the example of FIG. 1 illustrates five separate I/O connectors 14, it should be understood that in other examples, hemodynamic monitor 10 can include fewer than five I/O connectors or greater than five I/O connectors. In yet other examples, hemodynamic monitor may not include I/O connectors 14, but rather may communicate wirelessly with various peripheral devices.

As is further described below, hemodynamic monitor 10 includes one or more processors and computer-readable memory that stores post-induction hypotension prediction software code which is executable to produce a post-induction score representing a likelihood that a patient will experience a future hypotensive event after the beginning of administration/induction of anesthesia. For example, hemodynamic monitor 10 can receive sensed hemodynamic data representative of an arterial pressure waveform of the patient, such as via one or more hemodynamic sensors connected to hemodynamic monitor 10 via I/O connectors 14. Hemodynamic monitor 10 executes the post-induction hypotension prediction software code to determine, using the received hemodynamic data and extracted waveform features from the hemodynamic data, a post-induction score. The post-induction hypotension prediction software code is trained (as will be discussed below) to select the most indictive waveform features from the hemodynamic data and weight the waveform features using predictive risk model coefficients to return a post-induction score that most accurately represents a likelihood that the patient will experience a hypotensive event after the beginning of administration of anesthesia. The plurality of predictive risk model coefficients, as described in further detail below, can be determined based on a standard mean arterial pressure (MAP) threshold, such as 65 mmHg, or other defined pressure threshold, such as a relative drop in pressure from baseline (e.g., a twenty percent drop in MAP as compared to the pre-induction MAP).

For example, as illustrated in FIG. 1, hemodynamic monitor 10 can present a graphical user interface at display 12. Display 12 can also display an indication of the post-induction score. Display 12 can be a liquid crystal display (LCD), a light-emitting diode (LED) display, an organic light-emitting diode (OLED) display, or other display device suitable for providing information to users in graphical form. In some examples, such as the example of FIG. 1, display 12 can be a touch-sensitive and/or presence-sensitive display device configured to receive user input in the form of gestures, such as touch gestures, scroll gestures, zoom gestures, swipe gestures, or other gesture input. Hemodynamic monitor 10 presents control elements that enable user input by, for example, medical personnel.

Hemodynamic monitor 10 executes the post-induction hypotension prediction software code to determine the post-induction score representing a likelihood that a patient will experience a hypotensive event after the beginning of administration/induction of anesthesia using the waveform features and predictive risk model coefficients that were determined during training of the predictive risk model. Hemodynamic monitor 10 can invoke a sensory alarm, such as an audible alarm, a haptic alarm, or other sensory alarm in response to determining that the post-induction score satisfies predetermined criteria. Accordingly, hemodynamic monitor 10 can provide a warning to medical personnel of a predicted future post-induction hypotensive event of the patient prior to the administration of anesthesia and prior to the patient entering a hypotensive state. Hemodynamic monitor 10 can execute the post-induction hypotensive prediction software to preprocess the hemodynamic data to, for example, filter the sensed hemodynamic data to reduce noise that is not indicative of the arterial pressure waveform of the patient, identify individual heartbeat portions of the arterial pressure waveform with each individual heartbeat portion corresponding to a single heartbeat of the patient, and/or identify a dichrotic notch within one or more of the identified heartbeat portions.

Additionally, hemodynamic monitor 10 can be configured to receive patient demographic information/features, such as the age of the patient, the gender of the patient, the height and weight of the patient, and the patient physical status classification score. Then, the post-induction hypotension prediction software code can utilize the patent demographic features, along with the waveform features, to determine the post-induction score. The patient demographic information/features can be input into hemodynamic monitor 10 via display 12, can be received from a hospital server that contains the patient demographic information/features, can be transmitted to hemodynamic monitor 10 by patient monitoring equipment, or can be inputted into hemodynamic monitor 10 another way.

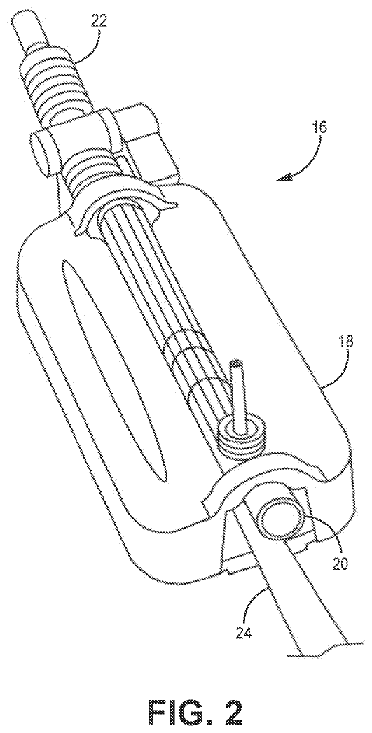

FIG. 2 is a perspective view of hemodynamic sensor 16 that can be attached to a patient for sensing hemodynamic data representative of arterial pressure of the patient. Hemodynamic sensor 16, illustrated in FIG. 2, is one example of a minimally invasive hemodynamic sensor that can be attached to the patient via, for example, a radial arterial catheter inserted into an arm of the patient. In other examples, hemodynamic sensor 16 can attached to the patient via a femoral arterial catheter inserted into a leg of the patient.

As illustrated in FIG. 2, hemodynamic sensor 16 includes housing 18, fluid input port 20, catheter-side fluid port 22, and I/O cable 24. Fluid input port 20 is configured to be connected via tubing or other hydraulic connection to a fluid source, such as a saline bag or other fluid input source. Catheter-side fluid port 22 is configured to be connected via tubing or other hydraulic connection to a catheter (e.g., a radial arterial catheter or a femoral arterial catheter) that is inserted into an arm of the patient (i.e., a radial arterial catheter) or a leg of the patient (i.e., a femoral arterial catheter). I/O cable 24 is configured to connect to hemodynamic monitor 10 via, for example, one or more of I/O connectors 14 (shown in FIG. 1). Housing 18 of hemodynamic sensor 16 encloses one or more pressure transducers, communication circuitry, processing circuitry, and corresponding electronic components to sense fluid pressure corresponding to arterial pressure of the patient that is transmitted to hemodynamic monitor 10 (shown in FIG. 1) via I/O cable 24.

In operation, a column of fluid (e.g., saline solution) is introduced from a fluid source (e.g., a saline bag) through hemodynamic sensor 16 via fluid input port 20 to catheter-side fluid port 22 toward the catheter inserted into the patient. Arterial pressure is communicated through the fluid column to pressure sensors located within housing 16 which sense the pressure of the fluid column. Hemodynamic sensor 16 translates the sensed pressure of the fluid column to an electrical signal via the pressure transducers and outputs the corresponding electrical signal to hemodynamic monitor 10 (shown in FIG. 1) via I/O cable 24. Hemodynamic sensor 16 therefore transmits analog sensor data (or a digital representation of the analog sensor data) to hemodynamic monitor 10 (shown in FIG. 1) that is representative of substantially continuous beat-to-beat monitoring of the arterial pressure of the patient. Hemodynamic sensor 16 is capable of measuring/collecting hemodynamic data representative of arterial pressure of the patient continuously for at least five minutes before the beginning of administration of anesthesia and then conveying that hemodynamic data to hemodynamic monitor 10 for use by the post-induction hypotension prediction software code to determine the post-induction score representing a likelihood that a patient will experience a hypotensive event after the beginning of administration/induction of anesthesia.

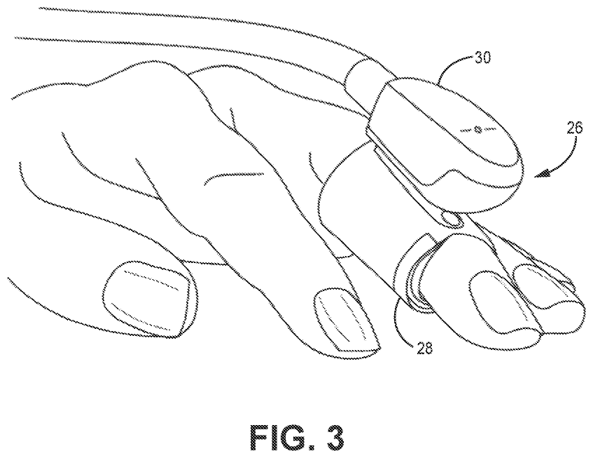

FIG. 3 is a perspective view of hemodynamic sensor 26 for sensing hemodynamic data representative of arterial pressure of a patient. Hemodynamic sensor 26, illustrated in FIG. 3, is one example of a non-invasive hemodynamic sensor that can be attached to the patient via one or more finger cuffs to sense data representative of arterial pressure of the patient. As illustrated in FIG. 3, hemodynamic sensor 26 includes inflatable finger cuff 28 and heart reference sensor 30. Inflatable finger cuff 28 includes an inflatable blood pressure bladder configured to inflate and deflate as controlled by a pressure controller (not illustrated) that is pneumatically connected to inflatable finger cuff 28. Inflatable finger cuff 28 also includes an optical (e.g., infrared) transmitter and an optical receiver that are electrically connected to the pressure controller (not illustrated) to measure the changing volume of the arteries under the cuff in the finger.

In operation, the pressure controller continually adjusts pressure within the finger cuff to maintain a constant volume of the arteries under the cuff in the finger (i.e., the unloaded volume of the arteries) as measured via the optical transmitter and optical receiver of inflatable finger cuff 28. The pressure applied by the pressure controller to continuously maintain the unloaded volume is representative of the blood pressure in the finger, and is communicated by the pressure controller to hemodynamic monitor 10 shown in FIG. 1. Heart reference sensor 30 measures the hydrostatic height difference between the level at which the finger is kept and the reference level for the pressure measurement, which typically is heart level. Accordingly, hemodynamic sensor 26 transmits sensor data that is representative of substantially continuous beat-to-beat monitoring of the arterial pressure waveform of the patient. Hemodynamic sensor 26 is capable of measuring/collecting hemodynamic data representative of arterial pressure of the patient continuously for a certain threshold time (e.g., at least five minutes before the beginning of administration of anesthesia and then conveying that hemodynamic data to hemodynamic monitor 10 for use by the post-induction hypotension prediction software code to determine the post-induction score representing a likelihood that a patient will experience a hypotensive event after the beginning of administration/induction of anesthesia.

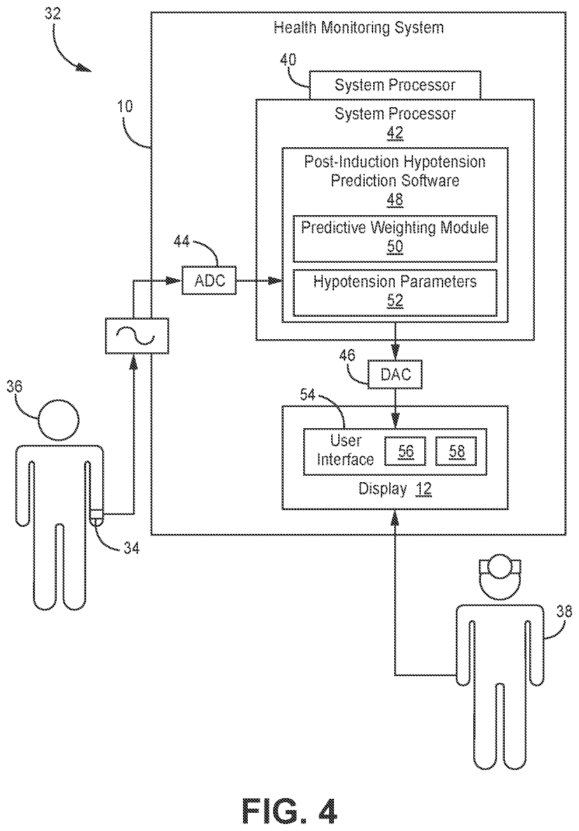

FIG. 4 is a block diagram of hemodynamic monitoring system 32 that determines a post-induction score representing a likelihood that a patient will experience a future hypotensive event after the beginning of administration/induction of anesthesia. As illustrated in FIG. 4, hemodynamic monitoring system 32 includes hemodynamic monitor 10 and hemodynamic sensor 34. Hemodynamic monitoring system 32 can be implemented within a patient care environment, such as an ICU, an OR, a surgery preparation environment where anesthesia is to be administered, or other patient care environment. As illustrated in FIG. 4, the patient care environment can include patient 36 and healthcare worker 38, such as an anesthesiologist, trained to utilize hemodynamic monitoring system 32.

Hemodynamic monitor 10, as described above with respect to FIG. 1, can be, for example, an integrated hardware unit including system processor 40, system memory 42, display 12, analog-to-digital (ADC) converter 44, and digital-to-analog (DAC) converter 46 all contained within a housing. In other examples, any one or more components and/or described functionality of hemodynamic monitor 10 can be distributed among multiple hardware units. For instance, in some examples, display 12 can be a separate display device that is remote from and operatively coupled with hemodynamic monitor 10. In general, though illustrated and described in the example of FIG. 4 as an integrated hardware unit, it should be understood that hemodynamic monitor 10 can include any combination of devices and components that are electrically, communicatively, or otherwise operatively connected to perform functionality attributed herein to hemodynamic monitor 10.

As illustrated in FIG. 4, system memory 42 stores post-induction hypotension prediction software code 48. Post-induction hypotension prediction software code 48 includes predictive weighting module 50 and hypotension parameters 52. Display 12 provides user interface 54, which includes control elements 56 that enable user interaction with hemodynamic monitor 10 and/or other components of hemodynamic monitoring system 32. User interface 54, as illustrated in FIG. 4, also provides sensory alarm 58 to provide warning to medical personnel if hemodynamic monitoring system 32 determines a post-induction score that is indicative of a strong likelihood that patient 36 will experience a hypotensive event after the beginning of administration/induction of anesthesia on patient 36, as is further described below.

Hemodynamic sensor 34 can be attached to patient 36 to sense hemodynamic data representative of an arterial pressure waveform of patient 36. Hemodynamic sensor 34 is operatively connected to hemodynamic monitor 10 (e.g., electrically and/or communicatively connected via wired or wireless connection, or both) to provide the sensed hemodynamic data to hemodynamic monitor 10. In some examples, hemodynamic sensor 34 provides the hemodynamic data representative of the arterial pressure waveform of patient 36 to hemodynamic monitor 10 as an analog signal, which is converted by ADC 44 to digital hemodynamic data representative of the arterial pressure waveform. In other examples, hemodynamic sensor 34 can provide the sensed hemodynamic data to hemodynamic monitor 10 in digital form, in which case hemodynamic monitor 10 may not include or utilize ADC 44. In yet other examples, hemodynamic sensor 34 can provide the hemodynamic data representative of the arterial pressure waveform of patient 36 to hemodynamic monitor 10 as an analog signal, which is analyzed in its analog form by hemodynamic monitor 10.

Hemodynamic sensor 34 can be a non-invasive or minimally invasive sensor attached to patient 36. For instance, hemodynamic sensor 34 can take the form of minimally invasive hemodynamic sensor 16 (shown in FIG. 2), non-invasive hemodynamic sensor 26 (shown in FIG. 3), or other minimally invasive or non-invasive hemodynamic sensor. In some examples, hemodynamic sensor 34 can be attached non-invasively at an extremity of patient 36, such as a wrist, an arm, a finger, an ankle, a toe, or other extremity of patient 36. As such, hemodynamic sensor 34 can take the form of a small, lightweight, and comfortable hemodynamic sensor suitable for extended wear by patient 36 to provide substantially continuous beat-to-beat monitoring of the arterial pressure of patient 36 over an extended period of time, such as minutes or hours. The hemodynamic data needed to determine the post-induction score is collected before patient 36 is administered/induced anesthesia, so hemodynamic sensor 34 should be monitoring (i.e., collecting hemodynamic data) patient 36 at least before the administration/induction of anesthesia. For example, hemodynamic sensor 34 should be monitoring/collecting hemodynamic data continuously for a threshold time period (e.g., at least five minutes) before the beginning of administration of anesthesia on patient 36.

In certain examples, hemodynamic sensor 34 can be configured to sense an arterial pressure of patient 36 in a minimally invasive manner. For instance, hemodynamic sensor 34 can be attached to patient 36 via a radial arterial catheter inserted into an arm of patient 36. In other examples, hemodynamic sensor 34 can be attached to patient 36 via a femoral arterial catheter inserted into a leg of patient 36. Such minimally invasive techniques can similarly enable hemodynamic sensor 34 to provide substantially continuous beat-to-beat monitoring of the arterial pressure of patient 36 over an extended period of time, such as minutes or hours.

System processor 40 is configured to execute post-induction hypotension prediction software code 48, which implements predictive weighting module 50 utilizing hypotension parameters 52 and risk model coefficients to produce a post-induction score representing a likelihood that patient 36 will experience a hypotensive event after the beginning of administration of anesthesia. Examples of system processor 40 can include any one or more of a microprocessor, a controller, a digital signal processor (DSP), an application specific integrated circuit (ASIC), a field-programmable gate array (FPGA), or other equivalent discrete or integrated logic circuitry.

System memory 42 can be configured to store information within hemodynamic monitor 10 during operation. System memory 42, in some examples, is described as computer-readable storage media. In some examples, a computer-readable storage medium can include a non-transitory medium. The term “non-transitory” can indicate that the storage medium is not embodied in a carrier wave or a propagated signal. In certain examples, a non-transitory storage medium can store data that can, over time, change (e.g., in RAM or cache). System memory 42 can include volatile and non-volatile computer-readable memories. Examples of volatile memories can include random access memories (RAM), dynamic random-access memories (DRAM), static random-access memories (SRAM), and other forms of volatile memories. Examples of non-volatile memories can include, for example, magnetic hard discs, optical discs, flash memories, or forms of electrically programmable memories (EPROM) or electrically erasable and programmable (EEPROM) memories.

Display 12 can be a liquid crystal display (LCD), a light-emitting diode (LED) display, an organic light-emitting diode (OLED) display, or other display device suitable for providing information to users in graphical form. User interface 54 can include graphical and/or physical control elements that enable user input to interact with hemodynamic monitor 10 and/or other components of hemodynamic monitoring system 32. In some examples, user interface 54 can take the form of a graphical user interface (GUI) that presents graphical control elements presented at, for example, a touch-sensitive and/or presence sensitive display screen of display 12. In such examples, user input can be received in the form of gesture input, such as touch gestures, scroll gestures, zoom gestures, or other gesture input. In certain examples, user interface 54 can take the form of and/or include physical control elements, such as a physical buttons, keys, knobs, or other physical control elements configured to receive user input to interact with components of hemodynamic monitoring system 32.

In operation, hemodynamic sensor 34 senses hemodynamic data representative of an arterial pressure waveform of patient 36 before the beginning of administration of anesthesia on patient 36. Hemodynamic sensor 34 provides the hemodynamic data (e.g., as analog sensor data), to hemodynamic monitor 10. ADC 44 converts the analog hemodynamic data to digital hemodynamic data representative of the arterial pressure waveform of the patient.

System processor 40 executes post-induction hypotension prediction software code 48 to determine, using the received hemodynamic data, a post-induction score representing a likelihood that patient 36 will experience a hypotensive event after the beginning of administration of anesthesia. For instance, system processor 40 can execute post-induction hypotension prediction software code 48 to extract multiple hypotension parameters 52. Hypotension parameters 52 can include one or multiple arterial pressure waveform features that are most indicative in predicting the likelihood of a post-induction hypotensive event. Hypotensive parameters 52 can also include patient demographic information that is most indicative in predicting the likelihood of a post-induction hypotensive event. Hypotension parameters 52 can further include differential and combinatorial parameters derived from the one or more waveform features, inter-relational parameters derived from series of one or more waveform features and patient demographic information, as is further described below.

Predictive weighting module 50 of hypotension prediction software code 48 selects the predictive risk model coefficients to weight hypotension parameters 52 to determine the most accurate post-induction score corresponding to the likelihood that patient 36 will experience a hypotension event after the beginning of administration. That is, predictive weighting module 50 applies a plurality of predictive risk model coefficients stored at system memory 42 to hypotension parameters 52 to produce the weighted combination resulting in the post-induction score. The predictive risk model coefficients can be determined via training operations (e.g., offline training) using machine learning or other techniques to minimize an error of the post-induction score outputted by the post-induction hypotension prediction software code 48 (e.g., aggregations of data from multiple patients) that define hypotension according to a MAP threshold for hypotension. That is, predictive risk model coefficients utilized by predictive weighting module 50 can be selected via training operations to minimize the error of the post-induction score determined by hypotension prediction software code 48 as predictive of a hypotension event occurring after the beginning of administration of anesthesia on patient 36. The error of the post-induction score to predict hypotension events occurring after administration of anesthesia can be evaluated with respect to positive and negative training data subsets (e.g., from subjects that experienced a hypotensive event and subjects that did not experience a hypotensive event after the administration of anesthesia) that define the occurrence of hypotension with respect to a MAP threshold, such as 65 mmHg or other pressure thresholds.

Post-induction hypotension prediction software code 48 can determine the post-induction score before the administration of anesthesia and continuously update the post-induction score as the anesthesia is being administered (and after the anesthesia is finished being administered), or the post-induction hypotension prediction software code 48 can determine the post-induction score once immediately before the beginning of administration of anesthesia such that the post-induction score is no longer calculated and remains static after originally determined at the time of beginning of administration of anesthesia.

System processor 40 executes post-induction hypotension prediction software code 48 and can invoke sensory alarm 58 via user interface 54 in response to determining that the post-induction score satisfies predetermined risk criteria, as is further described below. For instance, hypotension prediction software code 48 can invoke sensory alarm 58 to warn of a hypotension event predicted to occur, for example, within fifteen minutes after the beginning of administration of anesthesia. The post-induction score can be a normalized value between 0 and 1 (or between 0 and 100, or other normalized ranges) with, in some examples, a higher value representing a higher likelihood that patient 36 will experience a hypotensive event after beginning the administration of anesthesia and a lower value representing a lower likelihood that patient 36 will experience a hypotensive event after beginning the administration of anesthesia. Sensory alarm 58 can be configured to be invoked if, for example, the post-induction score is greater than 0.8 (when measured on a normalized scale of 0 to 1) or 80 (when measured on a normalized scale of 0 to 100). Sensory alarm 58 can be implemented as one or more of a visual alarm, an audible alarm, a haptic alarm, or other type of sensory alarm. For instance, sensory alarm 58 can be invoked as any combination of flashing and/or colored graphics shown by user interface 54 on display 12, display of the post-induction score via user interface 54 on display 12, a warning sound such as a siren or repeated tone, and a haptic alarm configured to cause hemodynamic monitor 10 to vibrate or otherwise deliver a physical impulse perceptible to healthcare worker 38 or other user.

Accordingly, hemodynamic monitor 10 provides a warning to medical personnel of a likelihood that patient 36 will experience a hypotensive event after the beginning of administration of anesthesia with the determination of the post-induction score and the potential warning occurring before the administration of anesthesia, thereby enabling timely and effective intervention to prevent (or mitigate) the predicted hypotension event that may occur after the administration of anesthesia. Intervention may include adapting or titrating the dose and speed of administration of anesthesia. Techniques described herein therefore increase the usability of hemodynamic monitor 10 by enabling hemodynamic monitor 10 to determine, before the administration of anesthesia, the likelihood that patient 36 will experience a hypotensive event after the administration of anesthesia, which may be difficult to accurately predict due to the unpredictable nature of the response of patient 36 to anesthesia.

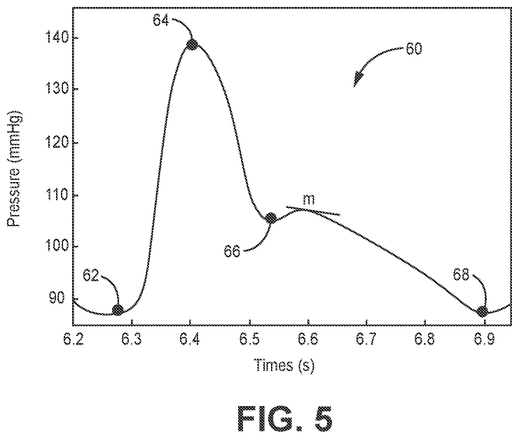

FIG. 5 is a graph illustrating an example trace of arterial pressure waveform 60 corresponding to hemodynamic data sensed by hemodynamic sensor 34 and received by hemodynamic monitor 10.

System processor 40 executes post-induction hypotension prediction software code 48 to determine hypotension parameters 52, which can be entirely or partly based on arterial pressure waveform 60. Predictive weighting module 50 applies predictive risk model coefficients to weight hypotension parameter 52 to determine an accurate post-induction score representing a likelihood that patient 36 will experience a hypotensive event after the beginning of administration of anesthesia.

Hemodynamic waveform 60 (e.g., represented via digital hemodynamic data) can include various indicia predictive of a future hypotension event for patient 36. FIG. 5 illustrates example indicia, such as the start of a heart beat (indicium 62), the maximum systolic pressure marking the end of systolic rise (indicium 64), the presence of the dicrotic notch marking the end of systolic decay (indicium 66), and the diastole of the heartbeat (indicium 68) of patient 36. Also shown in FIG. 5 is example slope “m” of arterial pressure waveform 60, though it should be understood that slope “m” is merely representative of multiple slopes that may be determined at multiple locations along arterial pressure waveform 60.

Additional indicia predictive of future hypotension for patient 36 can be extracted from hemodynamic waveform 60 by post-induction hypotension prediction software code 48 based on behavior of hemodynamic waveform 60 in various intervals, such as in the interval from the maximum systolic pressure at indicium 64 to the diastole at indicium 66, as well as the interval from the start of the heartbeat at indicium 62 to the diastole at indicium 66. The behavior of arterial pressure waveform 60 during intervals: 1) systolic rise 62-64, 2) systolic decay 64-66, 3) systolic phase 62-66, 4) diastolic phase 66-68, 5) interval 64-68, and 6) heartbeat interval 62-68, can be determined by post-induction hypotension prediction software code 48 by determining the area under the curve of hemodynamic waveform 60 and the standard deviation of hemodynamic waveform 60 in each of intervals 1-6 detailed above. The respective areas and standard deviations determined for intervals 1-6 can serve as additional indicia predictive of future hypotension for patient 36. Additional indicia predictive of future hypotension may include combinatorial features combining two of more individual features, or inter-relationship features describing relationships between short time series (e.g., over twenty seconds) of two or more individual features.

One example of an equation for determining the post-induction hypotension risk score is as follows:

$$Risk Score = \frac{100}{1 + {e}^{- ( {w}_{0} + \sum {w}_{i} {x}_{i} )}} for i = 1 , 2 , 3 , … , 13 ( total number of features in the model )$$

where, xᵢ's are the example arterial pressure waveform 60 features (examples listed below), wᵢ's are the corresponding feature weights (i.e., coefficients), and w₀ is a bias term.
x₁=The normalized area under the diastolic portion of the waveform
x₂=The mean of the diastolic portion of the waveform
x₃=The normalized area from the systolic max to the start of the next beat
x₄=The normalized area under the entire beat
x₅=The mean of the portion of the waveform extending from the systolic max to the start of next beat
x₆=The normalized area from the start of the beat to the systolic max
x₇=The mean of the portion of the waveform extending from the start of the beat to the systolic max
x₈=Mean arterial pressure
x₉=Diastolic pressure
x₁₀=The mean of the systolic portion of the waveform
x₁₁=Cardiac work index
x₁₂=The bias of the diastolic slope
x₁₃=The area from the start of the beat to the systolic max

The post-induction hypotension risk score can be determined using one, a selected number, or all of the arterial pressure waveform 60 features, and some examples of determining the post-induction hypotension risk score can include the use of other arterial pressure waveform 60 features other than those set out above.

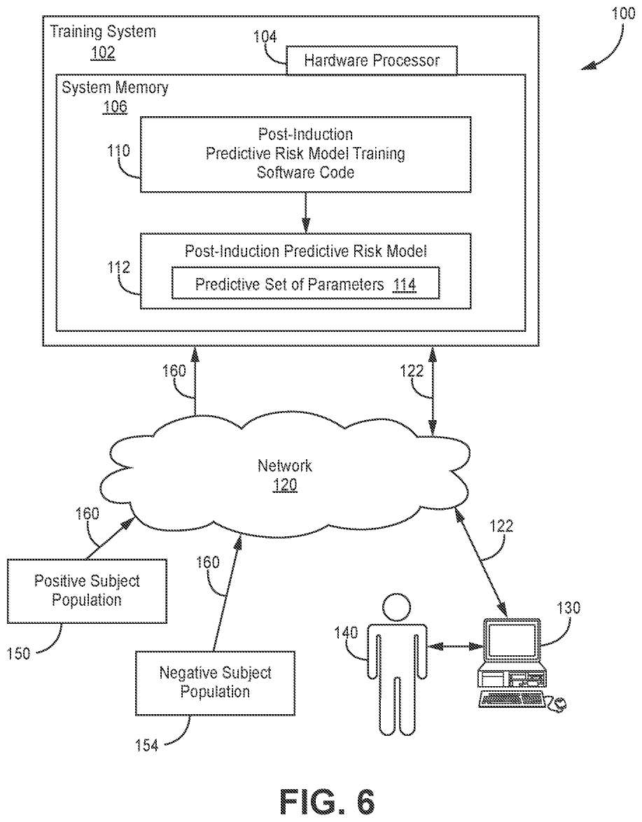

FIG. 6 shows a diagram illustrating example training system 102 for training a post-induction predictive risk model. Training system 102 is situated within communication environment 100 including communication network 120, client system 130, system user 140, population of positive subjects 150, and population of negative subjects 154. Training system 102 includes hardware processor 104 and system memory 106 for storing post-induction predictive risk model training software code 110. System memory 106 can include post-induction predictive risk model 112 including predictive set of parameters 114, which can be arterial pressure waveform features and/or patient demographic features/information. Network communication links 122 interactively connect client system 130 to training system 102 via communication network 120 and allow for the transmission of hemodynamic data 160 (which can include an arterial pressure waveform) from population of positive subjects 150 and population of negative subjects 154 to training system 102 via communication network 120.

System user 14; who may be a medical professional, health care working, or medical researcher; may utilize client system 130 to interact with training system 102 over communication network 120. For example, system user 140 may receive post-induction predictive risk model 112 (including predictive set of parameters 114) over communication network 120 and/or may download post-induction predictive risk model training software code 110 to client system 130 via communication network 120. In one implementation, training system 102 may correspond to one or more web servers with accessibility over a packet network, such as the internet. Alternatively, training system 102 may correspond to one or more servers supporting a local area network (LAN) or included in another type of limited distribution network.

Hardware processor 104 is configured to execute post-induction risk model training software code 110 to receive hemodynamic data 160 (which can include an arterial pressure waveform) of each subject of population of positive subjects 150 and each subject of population of negative subjects 154 with hemodynamic data 160 being collected for a period of time before the subjects begin receiving anesthesia, such as at least five minutes. In positive subjects 150, the patient experiences a hypotensive event after the beginning of administration of anesthesia, and in negative subjects 154, the patient does not experience a hypotensive event after the beginning of administration of anesthesia.

Hardware processor 104 is further configured to execute post-induction risk model training software code 110 to define hemodynamic data 160 sets for use in training the post-induction risk model and extract waveform features from the arterial pressure waveform (of the hemodynamic data 160 sets) of the positive subject 150. In addition, hardware processor 104 is configured to execute post-induction risk model training software code 110 to transform the waveform features from positive subjects 150 to a plurality of parameters characterizing the waveform features. Post-induction risk model training software code 110 then identifies, from the plurality of parameters, predictive set of parameters 114 enabling prediction that the patient will experience the hypotensive event after beginning the administration of anesthesia (e.g., identifies the waveform features that are most indicative in predicting a post-induction hypotensive event). The plurality of parameters characterizing the waveform features (extracted from the hemodynamic data) can be one, a combination of, or all of cardiac output, cardiac index, stroke volume, stroke volume index, pulse rate, systemic vascular resistance, systemic vascular resistance index, mean arterial pressure, baroreflex sensitivity measures, hemodynamic complexity measures, and frequency domain hemodynamic features. Additionally, the plurality of parameters can also include patient demographic information/features, such as an age, gender, height, weight, and physical status classification score of the positive subject

Identifying predictive set of parameter 114 from the plurality of parameters can include obtaining differential parameters (i.e., a second plurality of parameters) based on the plurality of parameters characterizing the waveform features and/or generating combinatorial parameters (a third plurality of combinatorial parameters) and/or generating inter-relationship parameters over short periods of time (a fourth plurality of inter-relational parameters) using the plurality of parameters characterizing the waveform features and the differential parameters. The differential parameters can be the same, partially the same, or different parameters than the plurality of parameters. Predictive set of parameters 114 can then be identified from the plurality of parameters, the differential parameters, the inter-relational parameters and the combinatorial parameters to select a reduced set of parameters that are most indicative of predicting a post-induction hypotensive event. The combinatorial parameters can be a power combination of all or a subset of the plurality of parameters and the differential parameters, and the power combinations can include integer powers from among, for example, negative two, negative one, positive one, and/or positive two.

Hardware processor 104 can also be configured to execute post-induction risk model training software code 110 to identify, from among the reduced set of parameters, predictive set of parameters 114 more correlated to the occurrence of a hypotensive event in the patient after the administration of anesthesia, thereby training post-induction predictive risk model 112. From the predictive set of parameters 114, hardware processor 104 can be configured to execute post-induction risk model training software code 110 to compute predictive risk model coefficients corresponding to the predictive set of parameters to minimize the error of the post-induction score outputted by post-induction predictive risk model 112, thereby further training post-induction predictive risk model 112 to minimize error.

Post-induction predictive risk model 112 (and post-induction risk model training software code 110), can be a machine learning model that is an artificial neural network model, a machine learning model that is a known nearest neighbor model, a machine learning model that utilizes linear regression to identify predictive set of parameters 114 and determine the predictive risk model coefficients, or another type of model for identifying predictive set of parameters 114 and determining the predictive risk model coefficients that most accurately represent the likelihood that a patient will experience a hypotensive event after the beginning of administration of anesthesia.

In some implementations, hardware processor 104 is configured to execute post-induction predictive risk model training software code 110 to display post-induction predictive risk model 112, the plurality of parameters characterizing hemodynamic data 160, and or predictive set of parameters 114 to system user 140 through display features available on client system 130. Additionally, hardware processor 104 is configured to execute post-induction predictive risk model training software code 110 to update or otherwise modify predictive set of parameters 114 and/or predictive risk model coefficients based on additional hemodynamic data 160 and/or patient demographic information/features received from one or more positive subjects of the population of positive subjects 150 and negative subjects of the population of negative subjects 154.

For example, training system 102 can receive additional hemodynamic data from one or more negative subjects from the population of negative subjects 154 (subjects that did not experience a hypotensive event after the administration of anesthesia). Hardware processor 104 can then execute post-induction predictive risk model training software code 110 to extract predictive set of parameters 114 (e.g., waveform features and/or patient demographic information/features) from the hemodynamic data with predictive set of parameters 114 being similar to predictive set of parameters 114 identified with respect to positive subject 150. Hardware processor 104 can then execute post-induction predictive risk model training software code 110 to then determine the post-induction score utilizing the same predictive risk model coefficients previously calculated and compare that post-induction score to a baseline post-induction score for a hypothetical negative subject that did not experience a hypotensive event after the beginning of administration of anesthesia. If the post-induction score is not within a margin of error of the baseline post-induction score, hardware processor 104 can then execute post-induction predictive risk model training software code 110 to alter predictive set of parameters 114 and the predictive risk model coefficients to more accurately predict the likelihood that a post-induction hypotensive event will occur, and training system 102 can then repeat the training steps with additional hemodynamic data from positive subjects 150 and/or negative subjects 154.

Although FIG. 6 shows post-inductions risk model 112 as residing in or otherwise a part of system memory 106, other configurations can include a post-induction predictive risk model that is copied to non-volatile storage (not shown in FIG. 6) or may be transmitted to client system 130 via communication network 120. Further, though client system 130 is shown as a personal computer in FIG. 6, such a configuration is provided merely as an example and other configurations may include client system 130 as or within a mobile communication device, such as a smartphone or tablet computer.

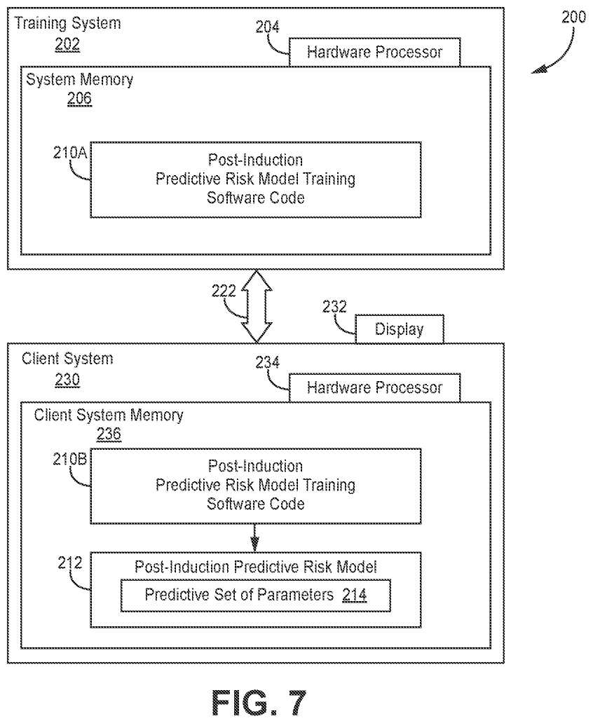

FIG. 7 shows a diagram illustrating another example system for training a post-induction hypotensive prediction model. FIG. 7 shows client system 230, which may be configured to train a post-induction predictive risk model. Communication network 200 in FIG. 7 can include client system 230 interactively connected to training system 202 over network communication link 222. Training system 202 includes hardware processor 204 and system memory 206, which can be configured to store post-induction predictive risk model training software code 210A. Client system 230 includes display 232, client hardware processor 234, and client system memory 236, which can be configured to store post-induction predictive risk model training software code 210B. Post-induction predictive risk model 212 includes predictive set of parameters 214.

Network communication link 222, training system 202, hardware processor 204, and system memory 206 correspond to network communication link 122, training system 102, hardware processor 104, and system memory 106 as shown in FIG. 6. In addition, post-induction predictive risk model training software code 210A in FIG. 7 corresponds to post-induction predictive risk model training software code 110 in FIG. 6 (i.e., post-induction predictive risk model training software code 210A may share any of the characteristics attributed to post-induction predictive risk model training software code 110).

Client system 230 corresponds to client system 130 as shown in FIG. 6, and post-induction predictive risk model training software code 210B corresponds to post-induction predictive risk model training software code 110 and/or 210A. Thus, post-induction predictive risk model training software code 210B and post-induction predictive risk model 112 including predictive set of parameters 214 may share any of the characteristics attributed to post-induction predictive risk model training software code 110 and post-induction predictive risk model 112 including predictive set of parameters 114 as shown in FIG. 6.

Example training system 202 can have post-induction predictive risk model training software code 210B located in client system memory 236, having been received from training system 202 via network communication link 222. One configuration includes network communication link 222 transferring post-induction predictive risk model training software code 210B over a packet network. Once transferred, (e.g., by being downloaded over network communication link 222), post-induction predictive risk model training software code 210B may be persistently stored in client system memory 236 and may be executed locally on client system 230 by client hardware processor 234

Client hardware processor 234 may be a central processing unit for client system 230, for example, so that client hardware processor 234 runs the operating system for client system 230 and executes post-induction predictive risk model training software code 210B. In example training system 202, system user 140 utilizing client system 230 can use post-induction predictive risk model training software code 210B on client system 230 to identify predictive set of parameters 214 and determine predictive risk model coefficients, thereby training predictive rise model 212.

Additionally, system user 140 can utilize post-induction predictive risk model training software code 210B on client system 230 to display post-induction predictive risk model 212, parameters characterizing hemodynamic data 160 and patient demographic information/features, and/or predictive set of parameters 214 on display 232. Display 232 can be a liquid crystal display (LCD), a light-emitting diode (LED) display, an organic light-emitting diode (OLED) display, or other display device suitable for providing information system user 140 in graphical form.

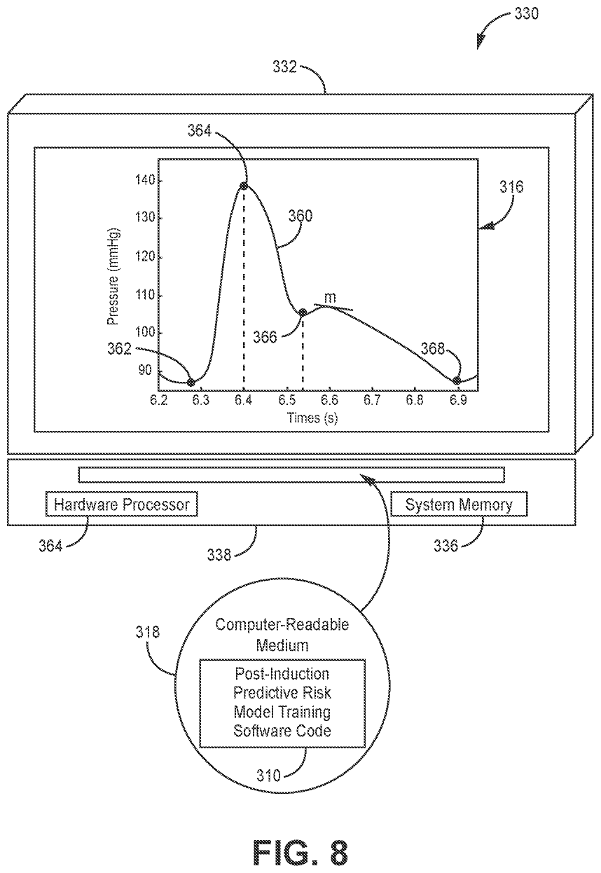

FIG. 8 is a diagram illustrating an example system and computer-readable medium including instructions for performing post-induction predictive risk model training. Training system 330 includes computer 338 having hardware processor 334 and system memory 336 interactively linked to display 332 (which can be similar to display 12 and display 232). Training system 220 including hardware processor 334 and system memory 336 correspond to training system 102 and client system 130 of FIG. 6 and training system 202 and client system 230 of FIG. 7.

Parameters 316 characterizing hemodynamic waveform 360 (and potentially patient demographic information/features) are shown on display 332 and include features 362, 364, 366, 368 and m of hemodynamic waveform 360. Features 362, 364, 366, 368 and m correspond to features 62, 64, 66, 68, and m of FIG. 5 and are from a positive subject (i.e., a subject that experienced a hypotensive event after the beginning of administration/induction of anesthesia). As described above with regards to FIG. 5, in addition to features 362, 364, 366, 368, and m of hemodynamic waveform 360, additional indicia predictive of future hypotension in a patient can be extracted from hemodynamic waveform 360 based on behavior of hemodynamic waveform 360 in various intervals 1-6 (set out with regards to FIG. 5 above). The features and intervals disclosed herein are provided only as exemplary and additional indicia predictive of future hypotension after the administration of anesthesia may be utilized.

Training system 330 includes computer-readable medium 318 with post-induction predictive risk model training software code 310 stored thereon. In some examples, computer-readable medium 318 is described as computer-readable storage media. In some examples, a computer-readable storage medium can include a non-transitory medium. The term “non-transitory” can indicate that the storage medium is not embodied in a carrier wave or a propagated signal. In certain examples, a non-transitory storage medium can store data that can, over time, change (e.g., in RAM or cache). Computer-readable medium 318 can include volatile and non-volatile computer-readable memories. Examples of volatile memories can include random access memories (RAM), dynamic random access memories (DRAM), static random access memories (SRAM), and other forms of volatile memories. Examples of non-volatile memories can include, for example, magnetic hard discs, optical discs, flash memories, or forms of electrically programmable memories (EPROM) or electrically erasable and programmable (EEPROM) memories.

As shown in FIG. 8, computer-readable medium 318 provides post-induction predictive risk model training software code 310 for execution by hardware processor 334 of computer 338. Post-induction predictive risk model training software code 310 corresponds to post-induction predictive risk model training software code 110, 210A, and 210B and is capable of performing all of the operations attributed to code 110, 210A, and 210B. For example, when executed by hardware processor 334, post-induction predictive risk model training software code 310 is configured to identify predictive set of parameters 114/214 and compute predictive risk model coefficients, thereby training post-induction predictive risk model 112/212.

As described above, a system for determining a post-induction score, such as a hemodynamic monitoring system, and related methods of determining the post-induction score and training a post-induction predictive risk model to determine the post-induction score produce a post-induction score that represents the likelihood that a patient will experience a hypotensive event after the beginning of administration of anesthesia. The post-induction score is determined based on hemodynamic data, such as arterial pressure waveform data collected by an arterial pressure sensor, during a period of time before the patient is administered/induced anesthesia. The post-induction score is determined and conveyed to a medical professional, such as an anesthesiologist, before the anesthesia is administered so that the medical professional has a warning that the patient is likely (or not likely) to experience a hypotensive event after the beginning of administration of anesthesia. The distinction that the system and methods predict the likelihood that the patient will experience a hypotensive event after the beginning of administration of anesthesia is important because other predictive systems and methods cannot accurately predict the likelihood of a hypotensive event when the patient is under the influence of anesthesia due to the generally unpredictable nature of the patient's response to the anesthesia.

The post-induction score is determined based on a weighted combination of a hypotension parameters, for example a predictive set of parameters that include waveform features extracted from the hemodynamic data and/or patient demographic information, that are predictive of the future hypotensive event. Risk coefficients that implement the weighting are selected based on a standard (or defined) mean arterial pressure (MAP) threshold for hypotension, such as a pressure of 65 millimeters of Mercury (mmHg) or other defined pressure threshold. The selection of risk coefficients and/or the predictive set of parameters can be accomplished via training (e.g., offline training) of the post-induction predictive risk model using machine learning or other techniques to minimize the error of the predictive risk model output (i.e., the post-induction score).

### Discussion of Possible Embodiments

The following are non-exclusive descriptions of possible embodiments of the present invention.

A method for determining a post-induction score that represents a prediction that a patient will experience a hypotensive event after beginning an administration of anesthesia. The method includes receiving, by a hemodynamic monitor prior to the administration of anesthesia on the patient, sensed hemodynamic data representative of an arterial pressure waveform of the patient; extracting, by the hemodynamic monitor, at least one waveform feature from the sensed hemodynamic data; determining, by the hemodynamic monitor based on the at least one waveform feature, the post-induction score that represents the likelihood that the patient will experience a hypotensive event after beginning the administration of anesthesia; and displaying an indication of the post-induction score.

The method of the preceding paragraph can optionally include, additionally and/or alternatively, any one or more of the following features, configurations, steps and/or additional components:

The hypotensive event is indicated by a mean arterial pressure of the patient dropping below a minimum mean arterial pressure level for a first threshold time period within a second threshold time period as measured from the beginning of the administration of anesthesia.

The minimum mean arterial pressure level is sixty-five millimeters Mercury.

The first threshold time period is one minute.

The second threshold time period is fifteen minutes.

Preprocessing the sensed hemodynamic data, wherein pre-processing the sensed hemodynamic data includes at least one of: filtering the sensed hemodynamic data to reduce noise that is not indicative of the arterial pressure waveform of the patient, identifying individual heartbeat portions of the arterial pressure waveform with each individual heartbeat portion corresponding to a single heartbeat of the patient, and identifying a dichrotic notch within one or more of the identified heartbeat portions.

Extracting the at least one waveform feature further includes identifying at least one of the following waveform features of the hemodynamic data: systolic rise, maximum systolic pressure, systolic decay, diastolic phase, heart rate, heart stroke volume, and cardiac output; and collecting at least one of the following measurements from the at least one waveform feature: a time, amplitude, area under the curve, slope, mean, and variance among identified individual heartbeat portions of the arterial pressure waveform.

Identifying a combination of at least two of the waveform features that are indicative of the post-induction score that represents the likelihood that the patient will experience a hypotensive event after beginning the administration of anesthesia.

Extracting the at least one waveform feature further includes identifying individual heartbeat portions of the arterial pressure waveform and collecting measurements from one or multiple individual heartbeat portions that include a variance among the individual heartbeat portions.

Receiving at least one patient demographic feature.

Determining, by the hemodynamic monitor based on the at least one waveform feature and the patient demographic feature, the post-induction score.

The at least one patient demographic feature includes at least one of: a patient age, patient gender, patient height, patient weight, and patient physical status classification score.

Invoking a sensory alarm in response to determining that the post-induction score satisfies threshold alarming criteria.

The post-induction score is a normalized value between a minimum normalized range value and a maximum normalized range value, with a higher value representing a higher likelihood that the patient will experience a hypotensive event after beginning the administration of anesthesia and a lower value representing a lower likelihood that the patient will experience a hypotensive event after beginning the administration of anesthesia.

The post-induction score is determined before the beginning of administration of anesthesia.

Determining the post-induction score is performed using a predictive risk model that utilizes a plurality of predictive risk coefficients identified based on the at least one waveform feature.

The predictive risk model and the plurality of predictive risk coefficients remain static after the hemodynamic monitor receives the sensed hemodynamic data.

Collecting, by an arterial pressure sensor, the sensed hemodynamic data representative of the arterial pressure waveform of the patient.

The collection of the sensed hemodynamic data is performed by a radial arterial catheter.

The collection of the sensed hemodynamic data is performed by a non-invasive hemodynamic sensor that is attached to the patient via a cuff.

Collecting the sensed hemodynamic data further includes collecting the sensed hemodynamic data from the patient continuously for a third time threshold period prior to the beginning of the administration of anesthesia.

The third time threshold period is at least five minutes.

A method for use by a system for training a predictive risk model to determine a post-induction score that represents a prediction that a patient will experience a hypotensive event after beginning an administration of anesthesia. The system including a hardware processor and predictive risk model training software code stored in a system memory with the hardware processor configured to execute the predictive risk model training software code. The method includes receiving hemodynamic data representing an arterial pressure waveform of a positive subject of a population of subjects including positive subjects that experienced a hypotensive event after beginning the administration of anesthesia; defining hemodynamic data sets for use in training the predictive risk model with the hemodynamic data sets including the arterial pressure waveform collected before the beginning of the administration of the anesthesia from the positive subject; extracting, by the predictive risk model training software code executed by the hardware processor, waveform features from the arterial pressure waveform of the positive subject; transforming, by the predictive risk model training software code executed by the hardware processor, the waveform features from the positive subject to a first plurality of parameters characterizing the waveform features; identifying, from the first plurality of parameters, a predictive set of parameters enabling prediction that the patient will experience the hypotensive event after beginning the administration of anesthesia; and computing predictive risk model coefficients to minimize an error of the post-induction score output by the predictive risk model, thereby training the predictive risk model.

The method of the preceding paragraph can optionally include, additionally and/or alternatively, any one or more of the following features, configurations, steps and/or additional components:

Storing the predictive risk model coefficients in the system memory.

The predictive set of parameters is identified by testing predictions of hypotensive events using each of the first plurality of parameters.

The predictive set of parameters is identified by having a measured correlation with the hypotensive event that satisfies a threshold correlation value.

The predictive set of parameters is identified by adding the parameters of the first plurality of parameters to a classification model or a regression model, or removing parameters of the first plurality of parameters from the classification model of the regression model.

The predictive set of parameters is identified utilizing a machine learning model.

The machine learning model is an artificial neural network model.

The machine learning model is a known nearest neighbor (KNN) model.

The predictive set of parameters is identified using logistic regression.

Transforming the waveform features to the first plurality of parameters further includes determining, from the arterial pressure waveform, on a heartbeat-by-heartbeat basis, indicia representative of one or more of: start of a heartbeat, maximum systolic pressure marking end of systolic rise, presence of a dicrotic notch marking end of systolic decay, diastole of the heartbeat, and slopes of the arterial pressure waveform; determining, from the arterial pressure waveform, one or more intervals from the group consisting of: systolic rise interval, systolic decay interval, systolic phase interval, diastolic phase interval, maximum systolic pressure to diastole interval, and heartbeat interval; and producing one or more parameters representing behavior of the arterial pressure waveform during one or more intervals, including one or more of areas under a curve of the arterial pressure waveform and standard deviations for the one or more intervals.

Obtaining, by the predictive risk model training software code executed by the hardware processor, a second plurality of parameters based on the first plurality of parameters; and analyzing, by the predictive risk model training software code executed by the hardware processor, the first plurality of parameters and the second plurality of parameters to identify a reduced set of parameters correlated with an occurrence of the hypotensive event, wherein identifying the predictive set of parameters enabling prediction that the patient will experience the hypotensive event includes identifying the predictive set from the first plurality of parameters and the second plurality of parameters.

Generating, by the predictive risk model training software code executed by the hardware processor, a third plurality of combinatorial parameters using the first plurality of parameters and the second plurality of parameters, wherein identifying the predictive set of parameters enabling prediction that the patient will experience the hypotensive event includes identifying the predictive set from the first plurality of parameters, the second plurality of parameters, and the third plurality of combinatorial parameters.

Each of the third plurality of combinatorial parameters comprises a power combination of a subset of the first plurality of parameters and the second plurality of parameter.

The power combination includes integer powers from among negative two, negative one, one, and two (−2, −1, 1, 2).

The plurality of first parameters includes at least one of: cardiac output, cardiac index, stroke volume, stroke volume index, pulse rate, systemic vascular resistance, systemic vascular resistance index, mean arterial pressure, baroreflex sensitivity measures, hemodynamic complexity measures, and frequency domain hemodynamic features.

Transmitting, by the hardware processor, the predictive risk model via a communication network to a client system.

The client system is a mobile communication device.

Outputting the hemodynamic data for visual display at a display device operatively coupled to the hemodynamic data.

Collecting the hemodynamic data from the positive subject prior to the beginning of administration of anesthesia.

Collecting the hemodynamic data further includes collecting the hemodynamic data from the positive subject continuously for a time threshold period prior to the beginning of the administration of anesthesia.

The time threshold period is at least five minutes.

Receiving demographic information of the positive subject; defining demographic information sets for use in training the predictive model; extracting, by the predictive risk model training software code executed by the hardware processor, at least one patient demographic feature from the demographic information of the positive subject; transforming the at least one demographic feature to be at least one parameter of the first plurality of parameters, wherein the demographic information includes at least one of: an age, gender, height, weight, and physical status classification score of the positive subject.

Receiving hemodynamic data of a negative subject of the population of subjects including negative subjects that do not experience a hypotensive event after beginning the administration of anesthesia; extracting waveform features from the arterial pressure waveform of the negative subject, the waveform features being similar features to the waveform features from the positive subject; determining the post-induction score utilizing the predictive risk model coefficients and the predictive risk model; and comparing the post-induction score to a baseline post-induction score for a hypothetical negative subject that did not experience a hypotensive event after beginning the administration of anesthesia.

If the post-induction score of the negative subject is not within a margin of error of the baseline post-induction score, repeating the steps of claim 1 utilizing hemodynamic data of another positive subject of the population of subjects to refine the predictive risk model coefficients to minimize the error of the post-induction score output by the predictive risk model.

The post-inductions score is a normalized value between 0 and 100 with a higher value representing a higher likelihood that the patient will experience a hypotensive event after beginning an administration of anesthesia and a lower value representing a lower likelihood that the patient will experience a hypotensive event after beginning the administration of anesthesia.

A system for determining a post-induction score that represents a prediction that a patient will experience a hypotensive event after beginning an administration of anesthesia. The system includes a hemodynamic sensor that collects sensed hemodynamic data representative of an arterial pressure waveform of the patient, the sensed hemodynamic data being collected from the patient prior to the administration of anesthesia; a system memory that stores post-induction hypotension prediction software code including a hypotension predictive risk model; and a hemodynamic monitor that includes a hardware processor that is configured to execute the post-induction hypotension prediction software code to: receive the sensed hemodynamic data from the hemodynamic sensor; extract at least one waveform feature from the sensed hemodynamic data; and determine, based on the at least one waveform feature, the post-induction score that represents the likelihood that the patient will experience a hypotensive event after beginning the administration of anesthesia by utilizing the hypotension prediction risk model.

The system of the preceding paragraph can optionally include, additionally and/or alternatively, any one or more of the following features, configurations, steps and/or additional components:

The hemodynamic sensor is an arterial pressure sensor.

The arterial pressure sensor is a radial arterial catheter.

The hemodynamic sensor is a non-invasive finger cuff.

The hardware processor is configured to execute the post-induction hypotension prediction software to pre-process the sensed hemodynamic data.

Extracting at least one waveform feature from the sensed hemodynamic data includes at least one of: filtering the sensed hemodynamic data to reduce noise that is not indicative of the arterial pressure waveform of the patient, identifying individual heartbeat portions of the arterial pressure waveform with each individual heartbeat portion corresponding to a single heartbeat of the patient, and identifying a dichrotic notch within one or more of the identified heartbeat portions.

Extracting at least one waveform feature from the sensed hemodynamic data further includes identifying a combination of at least two of the waveform features that are indicative of the post-induction score that represents the likelihood that the patient will experience a hypotensive event after beginning the administration of anesthesia.

Extracting at least one waveform feature from the sensed hemodynamic data further includes identifying individual heartbeat portions of the arterial pressure waveform and collecting measurements from one or multiple individual heartbeat portions that include a variance among the individual heartbeat portions.

The hemodynamic monitor that includes the hardware processor that is configured to execute the post-induction prediction software code to determine the post-induction score further utilizes predictive risk model coefficients identified based on the at least one waveform feature.

A display screen that is configured to output the post-induction score for visual display.

The display screen is configured to output for visual display at least one vital sign of the patient.

The hemodynamic monitor that includes the hardware processor is configured to transmit the post-induction score to a client system.

The client system is a mobile communication device.

The at least one waveform feature is stored in the system memory.

The hemodynamic monitor that includes the hardware processor is configured to execute the post-induction prediction software code to determine the post-induction score before the beginning of administration of anesthesia and the display screen is configured to output for visual display the post-induction score before the beginning of administration of anesthesia.

The hemodynamic sensor communicates the sensed hemodynamic data to the hemodynamic monitor for use by the hypotension prediction software code.

The hemodynamic sensor is in wired communication with the hemodynamic monitor.

The hemodynamic sensor is in wireless communication with the hemodynamic monitor.

A housing within which the system memory and the hemodynamic monitor that includes the hardware processor are contained.

Patient monitoring equipment that contains at least one patient demographic feature of the patient and transmits the at least one patient demographic feature to the hemodynamic monitor.

The hemodynamic monitor with the hardware processor that is configured to execute the post-induction hypotension prediction software code is also capable of: receiving the at least one patient demographic feature from the patient monitoring equipment and determining, based on the at least one waveform feature and the at least one patient demographic feature, the post-induction score.

The at least one patient demographic feature includes at least one of: a patient age, patient gender, patient height, patient weight, and patient physical status classification score.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A system for determining a post-induction score that represents a prediction that a patient will experience a hypotensive event after beginning an administration of anesthesia, the system comprising:
a hemodynamic sensor that collects sensed hemodynamic data representative of an arterial pressure waveform of the patient, the sensed hemodynamic data being collected from the patient prior to the administration of anesthesia;
a system memory that stores post-induction hypotension prediction software code including a hypotension predictive risk model; and
a hemodynamic monitor that includes a hardware processor that is configured to execute the post-induction hypotension prediction software code to:
receive the sensed hemodynamic data from the hemodynamic sensor;
extract at least one waveform feature from the sensed hemodynamic data; and
determine, based on the at least one waveform feature, the post-induction score that represents the likelihood that the patient will experience a hypotensive event after beginning the administration of anesthesia by utilizing the hypotension prediction risk model,
wherein the at least one waveform feature includes at least one of the following features: a normalized area under a diastolic portion of the arterial pressure waveform, a mean of the diastolic portion of the arterial pressure waveform, a normalized area from a systolic maximum to a start of a next beat of a heart of the patient, a normalized area under the entire beat, a mean of a portion of the arterial pressure waveform extending from the systolic maximum to the start of the next beat, a normalized area from the start of the beat to the systolic maximum, a mean of a portion of the arterial pressure waveform extending from the start of the beat to the systolic maximum, a mean arterial pressure, a diastolic pressure, a mean of a systolic portion of the arterial pressure waveform, a cardiac work index, a bias of a diastolic slope, and an area from the start of the beat to the systolic maximum.

2. The system of claim 1, wherein the hemodynamic sensor is an arterial pressure sensor.

3. The system of claim 2, wherein the arterial pressure sensor is a radial arterial catheter.

4. The system of claim 1, wherein the hemodynamic sensor is a non-invasive finger cuff.

5. The system of claim 1, wherein the hardware processor is configured to execute the post-induction hypotension prediction software to pre-process the sensed hemodynamic data.

6. The system of claim 1, wherein extracting at least one waveform feature from the sensed hemodynamic data includes at least one of: filtering the sensed hemodynamic data to reduce noise that is not indicative of the arterial pressure waveform of the patient, identifying individual heartbeat portions of the arterial pressure waveform with each individual heartbeat portion corresponding to a single heartbeat of the patient, and identifying a dichrotic notch within one or more of the identified heartbeat portions.

7. The system of claim 6, wherein extracting at least one waveform feature from the sensed hemodynamic data further includes identifying a combination of at least two of the waveform features that are indicative of the post-induction score that represents the likelihood that the patient will experience a hypotensive event after beginning the administration of anesthesia.

8. The system of claim 6, wherein extracting at least one waveform feature from the sensed hemodynamic data further includes identifying individual heartbeat portions of the arterial pressure waveform and collecting measurements from one or multiple individual heartbeat portions that include a variance among the individual heartbeat portions.

9. The system of claim 1, wherein the hemodynamic monitor that includes the hardware processor that is configured to execute the post-induction prediction software code to determine the post-induction score further utilizes predictive risk model coefficients identified based on the at least one waveform feature.

10. The system of claim 1, and further comprising a display screen that is configured to output the post-induction score for visual display.

11. The system of claim 10, wherein the display screen is configured to output for visual display at least one vital sign of the patient.

12. The system of claim 10, wherein the hemodynamic monitor that includes the hardware processor is configured to transmit the post-induction score to a client system.

13. The system of claim 12, wherein the client system is a mobile communication device.

14. The system of claim 1, wherein the at least one waveform feature is stored in the system memory.

15. The system of claim 1, wherein the hemodynamic monitor that includes the hardware processor is configured to execute the post-induction prediction software code to determine the post-induction score before the beginning of administration of anesthesia.

16. The system of claim 1, wherein the hemodynamic sensor communicates the sensed hemodynamic data to the hemodynamic monitor for use by the hypotension prediction software code.

17. The system of claim 1, and further comprising a housing within which the system memory and the hemodynamic monitor that includes the hardware processor are contained.

18. The system of claim 1, and further comprising patient monitoring equipment that contains at least one patient demographic feature of the patient and transmits the at least one patient demographic feature to the hemodynamic monitor.

19. The system of claim 18, wherein the hemodynamic monitor with the hardware processor that is configured to execute the post-induction hypotension prediction software code is also capable of:
receiving the at least one patient demographic feature from the patient monitoring equipment; and
determining, based on the at least one waveform feature and the at least one patient demographic feature, the post-induction score.

20. The system of claim 19, wherein the at least one patient demographic feature includes at least one of: a patient age, patient gender, patient height, patient weight, and patient physical status classification score.
